(19) United States
(12) Patent Application Publication
(10) Pub. No.: US 2001/0049827 A1
(43) Pub. Date: Dec. 6, 2001

(54) METHOD FOR PROVIDING PATHOGEN-FREE PORCINE TISSUE SUITABLE FOR HUMAN TRANSPLANTATION

(75) Inventors: RICHARD HUNTER, PRINCETON, NJ (US); E. MICHAEL EGAN, BROOKLINE, MA (US)

Correspondence Address: FISH AND RICHARDSON, 225 FRANKLIN STREET, BOSTON, MA, 021102804

(73) Assignee: DIACRIN, INC.

(21) Appl. No.: 08/906,009

(22) Filed: Aug. 4, 1997

Related U.S. Application Data
(63) Continuation of application No. 08/630,282, filed on Apr. 10, 1996, now abandoned.

(51) Int. Cl. A01K 67/00; A01K 67/033; A61K 48/00

(52) U.S. Cl. 800/8; 435/178; 424/93.1; 424/93.7; 424/93.21

(57) ABSTRACT

Methods for providing a pathogen-free pig or pig fetus as a donor of tissue, cells and/or organs to a recipient human. Animals are free of zoonotic pathogens. When fetal tissues are used for transplantation, donor animals are free from zoonotic pathogens, pathogens able to cross the placental barrier, and tissue-specific pathogens, e.g., neurotropic pathogens. Tissues, cells and organs from pigs free of the above-listed pathogens are suitable for transplantation into humans, include fetal neuronal cells for treatment of Parkinson's disease and islet cells for treatment of islet insufficiency-related diseases.

### BACKGROUND OF THE INVENTION

This invention relates to the production and use of animals as a source of tissues, cells and organs for transplantation to a human recipient.

Non-human animals are a potential source of donor tissue, cells and organs for transplantation into human recipients (xenografts). A major factor in achieving successful transplantation is the prevention of graft rejection. Long term administration of immunosuppressive drugs has been found to cause increased susceptibility to infection, renal failure, hypertension, and tumor growth. More recent methods of preventing graft rejection include modifying, masking or eliminating antigens capable of causing a T-lymphocyte-mediated response in the human host (e.g., U.S. Pat. No. 5,283,058).

Recently developed methods for disease treatment include transplantation of specific cells or tissue to a host in need thereof. For example, islet cell transplantation has been successfully performed in animals made diabetic by prior treatment with drugs which destroy pancreatic β cells. Successful transplantation in these animals has been shown to restore normal blood glucose regulation. Recent techniques have been developed for intracerebral transplantation of human fetal dopaminergic neurons in the treatment of Parkinson's disease. Transplantation of fetal neurons from human tissue requires the collection and dissection of tens of human fetuses in various states of surgical disruption. Donors are screened for transmissible diseases, but many human pathogens could escape detection under these relatively uncontrolled collection schemes.

The use of animals as a source of tissue for transplantation into humans would greatly increase the number of needy patients who could be treated for such diseases as Parkinson's disease, diabetes, Huntington's disease, focal epilepsy, cognitive disorders, familial hypercholesterolemia, acute and chronic liver disease, and coronary and congestive heart disease. Swine are good candidates for use as human tissue and organ donors for many reasons, including similarities in physiological function, similarity in secreted products such as insulin, and size. However, the use of unscreened animals as tissue and organ donors raises the dangerous potential of transmission of disease-causing pathogens from the donor animal to the human recipient (Chapman et al. (1995) N. Engl. J. Med. 333:1498-1501).

The only known program designed to improve pig quality is the National Specific Pathogen Free (SPF) program. The national SPF program is designed to ensure the health status of pigs used for food production and replacement stock (1. Rules and Regulations (1992) and 2.The Clean Power Sources (1994) National SPF Swine Accrediting Agency, Inc., Conrad, Iowa). Pigs entering the program are selected from controlled sources, and monitored by routine testing and controlled husbandry procedures. As a result of the program, pig mortality is lower, pigs have an improved feed efficiency and grow at a faster rate and with a more consistent rate of weight gain, with reduced antibiotic and veterinary expenses. The SPF program results in increased profitability of pig production and provides disease-controlled seedstock to commercial pig producers.

SPF accredited pigs can originate from approved laboratories where pigs are gnotobiotically derived and raised, or from accredited primary and secondary herds. A primary SPF herd is a closed herd, and all additions to the herd are through laboratory swine, embryo transfer or artificial insemination, and must be accompanied by a certificate issued by the laboratory. A secondary SPF herd is one that originates from an approved laboratory.

The SPF program monitors herds every 90 days, by farm and slaughter inspections for the symptoms of the following seven diseases: mange, pseudorabies, lice, swine dysentery, turbinate atrophy and/or snout distortion, pneumonic lesion, and brucellosis. Farm inspections are conducted by a licensed veterinarian who completes a health report on the animals. The pigs are serologically tested to show they are free of pseudorabies and brucellosis infection. Herd management practices also address shower-in/shower-out, double fencing, rodent and bird control, distance from other pigs, population density, cleanness of the operation, and air quality. Evidence of swine dysentery, lice/mange infestation, or pseudorabies results in the inspector placing the herd “on hold” status, and notification of the SPF office. Herds having clinical evidence of pneumonia and/or atrophic rhinitis (diseases that may lead to turbinate atrophy) are classified as health-controlled herds, and must pass two or more consecutive inspections before receiving SPF accreditation.

Generally, prior art approaches to providing animal tissue suitable for transplantation into humans involves the use of genetically-defined gnotobiotic animals raised in a sterile environment. This approach entails high production costs and low animal fertility rates. The present invention provides animal tissue suitable for transplantation into humans without these associated problems.

### SUMMARY OF THE INVENTION

This invention features methods for providing, as a donor of tissues, cells, and/or organs to a recipient human patient, a pig which is free from pathogens, any of which could be deleterious if passed from the donor tissue or cells to the recipient human patient.

Initially, a pig is determined to be free of specific pathogens by testing the pig for those pathogens and selecting an animal which is free from all of these pathogens. Each pig, after testing negative for all screened pathogens, is maintained with at least one, and preferably four or more, other pigs which are also free of those pathogens, in an enclosure under conditions which ensure that they remain essentially free from all of the screened pathogens.

Accordingly, in one aspect, the invention features an enclosure containing two or more pigs essentially free from pathogens. In a specific embodiment, the invention features an enclosure containing two or more pigs essentially free from zoonotic pathogens. Zoonotic pathogens include toxoplasma, brucella, listeria, mycobacterium TB, leptospirillum, rabies virus, pseudorabies virus, encephalomyocarditis virus, swine influenza type A virus, transmissible gastroenteritis virus, and vesicular stomatitis virus.

The invention features a method of producing and maintaining a pig essentially free of pathogens as a source of tissues, cells or organs for transplantation into a human recipient by selecting a pig free of pathogens and maintaining the pig in an enclosure under conditions in which the pig remains essentially free of pathogens. In a specific embodiment of the invention, the pigs are essentially free of zoonotic pathogens.

The invention provides tissues, cells or organs for transplantation into a human subject which are essentially free of zoonotic pathogens and pathogens affecting the specific tissues, cells, or organs to be transplanted.

In one embodiment, pancreatic islet cells free of zoonotic pathogens and pathogens affecting pancreatic islet cells are provided for transplantation into a human patient for treatment of islet insufficiency-related diseases, including diabetes. Pancreatic islet cells are specifically screened for the following pathogens: toxoplasma, brucella, listeria, mycobacterium TB, leptospirillum, rabies virus, pseudorabies virus, encephalomyocarditis virus, swine influenza type A virus, transmissible gastroenteritis virus, vesicular stomatitis virus, eperythrozoon suis, eperythrozoon parvum, haemophilus suis, mycoplasma hyopneumonia, porcine respiratory reproductive syndrome, parvovirus, teschen (porcine polio virus), hemagglutinating encephalomyocarditis, suipoxvirus, adenovirus, and bovine viral diarrhea virus. Further, parasites that could be present in the pancreas such as ascarids and echinococcus are screened by gross examination of the organ and by fecal examination, and bacteria which may infect the pancreas, such as salmonella and clostridium, are detected by culturing of blood and liver samples, and by gross pathological examination.

In another embodiment, hepatocytes free of zoonotic pathogens and pathogens affecting liver cells are provided for transplantation into a human patient for treatment of liver failure or liver insufficiency-related conditions, including insufficient liver function and familial hypercholesterolemia. Liver tissue is screened similarly to the pancreatic screening described above.

The invention includes the use of natural animals, as well as transgenic animals.

Included as a source of donor cells, tissues and organs are fetuses from pigs essentially free of zoonotic pathogens, pathogens capable of crossing the placental barrier, and pathogens affecting the fetal tissues, cells, or organs to be transplanted. In one embodiment, porcine fetal neuronal cells from fetuses harvested from pigs essentially free from zoonotic pathogens, pathogens capable of crossing the placental barrier, and neurotropic pathogens are provided for transplantation into a human patient for treatment of a neurodegenerative disease. Pathogens capable of crossing the placental barrier include toxoplasma, eperythrozoon suis, brucella, listeria, leptospirillum, mycoplasma hyopneumonia, porcine respiratory reproductive syndrome virus, rabies, pseudorabies, parvovirus, swine vesicular disease virus, teschen (porcine polio virus), hemagglutinating encephalomyocarditis virus, suipoxvirus, and swine influenza type A virus. Neurotropic pathogens include toxoplasma, listeria, rabies virus, pseudorabies virus, parvovirus, encephalomyocarditis virus, swine vesicular disease, teschen (porcine polio virus), hemagglutinating encephalomyocarditis, and adenovirus.

In addition, the invention provides fetal islet cells for treatment of Type I or Type II diabetes, and fetal cardiac myocytes for treatment of cardiac diseases. Fetal islet cells are obtained from fetuses harvested from pigs essentially free from zoonotic pathogens, pathogens capable of crossing the placental barrier, and pathogens affecting islet cells. Fetal cardiac myocytes are obtained from fetuses harvested from pigs essentially free from zoonotic pathogens, pathogens capable of crossing the placental barrier, and pathogens affecting cardiac myocytes, for example, encephalomyocarditis.

Described herein in the Examples are specific tests for viruses, bacteria, mycoplasma, and other parasites or pathogens which are zoonotic, capable of crossing the placental barrier (in the case of fetal cells, tissues or organs) and/or which adversely affect the cell/tissues/organ to be transplanted. The described tests screen for the following pathogens: adenovirus, bovine viral diarrhea, encephalomyocarditis virus, hemagglutinating encephalomyocarditis, parvovirus, porcine respiratory reproductive syndrome virus, pseudorabies, rabies, suipoxvirus, swine influenza type A, swine vesicular disease, teschen disease, transmissible gastroenteritis virus, vesicular stomatitis, brucella, clostridium, haemophilus suis, leptospirillum, listeria, mycobacterium TB, salmonella, ascarids, echinococcus, eperythrozoon parvum, eperythrozoon suis, mycoplasma hyopneumonia, and toxoplasma. The invention encompasses the use of these tests, as well as variations thereof and other tests able to identify the above-named organisms. It will be understood by those skilled in the art that the methods herein described are generally applicable, and the general applicability of the described methods apply beyond the above listed pathogens and pathogen screens to pathogens presently not known and to screening methods not yet available, for example, retroviral screens, which will be discovered and developed in the future.

Other features and advantages of the invention will be apparent from the following description of the preferred embodiments thereof, and from the claims.

### DETAILED DESCRIPTION

Reference will now be made in detail to useful embodiments of the invention, which, together with the following examples and claims, serve to explain the principles of the invention. It is to be understood that this invention is not limited to the specific examples described, and as such may, of course, vary. It is also to be understood that the terminology used herein is with the purpose of describing particular embodiments only, and is not intended to limit the scope of the invention which will be limited only by the appended claims.

Unless defined otherwise, all technical and scientific terms used herein have the same meaning as commonly understood by one of ordinary skill in the art to which this invention belongs. All publications mentioned herein are incorporated herein by reference to describe and disclose specific information for which the reference was cited in connection with.

Definitions

By the term “pathogen” is meant any virus, microorganism, or other substance causing disease.

By the term “essentially pathogen free” is meant an animal or tissue from an animal or herd which tests negative for the presence of pathogens. Described below are tests for screening porcine pathogens including parasites such as ascarids, echinococcus, toxoplasma, eperythrozoon suis, and eperythrozoon parvum, bacteria such as brucella, listeria, mycobacterium TB, salmonella, clostridium, leptospirillum, and haemophilus suis, mycoplasma such as mycoplasma hyopneumonia, and viruses such as porcine respiratory reproductive syndrome virus, rabies virus, pseudorabies virus, parvovirus, encephalomyocarditis virus, swine vesicular disease virus, teschen (porcine polio virus), hemagglutinating encephalomyocarditis virus, suipoxvirus, swine influenza type A virus, adenovirus, transmissible gastroenteritis virus, bovine viral diarrhea, and vesicular stomatitis virus.

By the term “zoonotic pathogen” is meant a pathogen which is transmittable between species, e.g., from pigs to humans. Zoonotic pathogens include toxoplasma, brucella, listeria, mycobacterium TB, leptospirillum, rabies virus, pseudorabies virus, encephalomyocarditis virus, swine influenza type A virus, transmissible gastroenteritis virus, and vesicular stomatitis virus.

By the term “a pathogen capable of crossing the placental barrier” is meant a pathogen which is transmittable from a mother to a fetus. Pathogens capable of crossing the placental barrier include toxoplasma, eperythrozoon suis, brucella, listeria, leptospirillum, mycoplasma hyopneumonia, porcine respiratory reproductive syndrome virus, rabies, pseudorabies, parvovirus, swine vesicular disease virus, teschen (porcine polio virus), hemagglutinating encephalomyocarditis virus, suipoxvirus, swine influenza type A virus.

By the term “neurotropic pathogen” is meant a pathogen which may infect neural tissue. Pathogens which infect neural tissue include toxoplasma, listeria, rabies, pseudorabies, parvovirus, encephalomyocarditis virus, swine vesicular disease, teschen (porcine polio virus), hemagglutinating encephalomyocarditis, and adenovirus.

By the term “neurodegenerative disease” is meant any pathological state involving neuronal degeneration, including Parkinson's disease, Huntington's disease, stroke, Alzheimer's disease, amyotrophic lateral sclerosis (ALS), cognitive diseases, spinal cord injury, focal epilepsy, and other conditions and diseases which may be treated by transplantation of appropriate healthy neuronal cells. For example, in one embodiment, the invention provides dopaminergic porcine fetal neural tissue free of the above-listed pathogens for clinical transplantation into humans suffering from Parkinson's disease.

By the terms “tissues”, “cells”, or “organs” are meant any collection of similar cells and the intercellular substances surrounding them, a single cell, or any part of a body exercising a specific function, which can be transplanted from a donor animal into a recipient subject.

Porcine Donors, Tissues and Cells

The use of animal sources for transplantable tissues and cells provides a solution to the scarcity of available donor tissue for human patients in need. It is of critical importance that transplantable tissues be free of pathogens capable of infecting or transmitting a disease to the recipient host. In order to provide a readily available source of consistently high quality cells, tissues and organs, and decrease the chance for transmission of disease, the present invention provides methods of producing and maintaining porcine donors for safe xenotransplantation of tissues, cells, and organs into humans.

Animals herein described may be employed as a source of a wide variety of cells, tissues, and organs suitable for transplant into a human host. Such cells, tissues and organs include fetal neuronal cells for transplantation into patients suffering from Huntington's disease, Parkinson's disease, stroke, Alzheimer's disease, focal epilepsy, cognitive disorders, and spinal cord injuries; islets or islet cells for transplantation into patients suffering from Type I or Type II diabetes; hepatocytes for treatment of familial hypercholesterolemia, liver failure, and liver insufficiency; and fetal cardiac myocytes for the treatment of cardiac disease. The invention ensures the safety and availability of porcine fetal neural cells for clinical transplantation into humans.

The invention includes the use of natural pathogen-free animals, as well as transgenic pathogen-free animals. Such transgenic animals are made by standard transgenic techniques known to the art. The transgenic pathogen-free animal of the invention is capable of expressing one or more exogenous DNA sequences in cells of all organs and tissues. The exogenous DNA sequence may be expressed constitutively, or be under the control of an inducible promoter which can be selectively activated to express an exogenous DNA sequence.

Pig or pig fetal cells, tissues and/or organs may be employed for transplantation into a recipient host in accordance with procedures for minimizing and/or eliminating an adverse immunoresponse. Thus, for example, the cells, tissues and/or organs may be masked by the use of the F(ab′)2 portion of an antibody directed against porcine Class I major histocompatibility antigen. Such a masking technique is described in U.S. Pat. No. 5,283,058, hereby incorporated by reference.

Animal Sources

Pigs were screened and selected from several reputable major swine producers in the United States and from a purebred Yorkshire pig herd maintained by Tufts University School of Veterinary Medicine (TUSVM), North Grafton, Mass. Pigs from each source were tested for evidence of infection by toxoplasma, eperythrozoon, brucella, listeria, mycobacterium TB, leptospirillum, haemophilus suis, mycoplasma hyopneumonia, porcine respiratory reproductive syndrome, rabies, pseudorabies, parvovirus, encephalomyocarditis virus, swine vesicular disease, teschen disease (porcine polio virus), hemagglutinating encephalomyocarditis, suipoxvirus, swine influenza type A, adenovirus, transmissible gastroenteritis virus, bovine viral diarrhea, and vesicular stomatitis virus. The results indicated that a fairly high percentage (but not all) of the pigs from the TUSVM were free from infection, while many of the pigs of other herds showed evidence of infection with various pathogens, including porcine respiratory reproductive syndrome virus (PRRS) and encephalomyocarditis virus (EMC).

Animal Testing and Maintenance Procedures

Prior to entering the donor qualification program, animals are moved away from the herd and quarantined in a controlled box stall. The animals are tested for evidence of infection by hematology (CBC with blood smear), serology, parasitology, and TB testing. Pathogens present in the U.S. and which are zoonotic are specifically tested. A record of routine clinical observations is kept for each animal starting at the time of quarantine. Animals are then tested for tuberculosis and fecal parasites, immunized against parvovirus, erysipelas, leptospira, and treated with the FDA approved deworming agent, Ivermectin.

Animals that have passed the prescreening tests, and have been treated and immunized as described above, are moved to an isolation room. The air pressure of this room is kept positive with a high efficiency particulate air (HEPA)-filtered, temperature and humidity controlled air handling system. Pigs are maintained following a carefully documented husbandry procedure herein described. Personnel can only enter the isolation room through a gowning room, must not have performed any other animal or animal-related work in that day, or must shower and change clothing before entry. When fetal tissue is to be generated, the donor gilts bred by artificial insemination are sacrificed for harvest.

Blood and fecal samples are collected from the animal. The blood is used for blood culture, a complete blood count and blood smear, and to test for viral contamination. Microscopic examination of blood smears reveals the presence of eperythrozoon suis and eperythrozoon parvum. Blood culture will reveal the presence of bacterial or fungal pathogens such as brucella, listeria, and haemophilus suis. The complete blood count can indicate general pathological conditions, including infection. The test for viral contamination has been validated to detect viruses which are zoonotic, including porcine respiratory reproductive syndrome virus, rabies virus, pseudorabies virus, encephalomyocarditis virus, swine influenza type A virus, transmissible gastroenteritis virus, and vesicular stomatitis virus.

The method of viral co-cultivation uses five sentinel cell lines (2 porcine, 1 monkey, and 2 human). After inoculating cells with blood samples, the sentinel cells are examined for cytopathic effect. Specific viral pathogens are detected qualitatively by the method of fluorescent antibody stain, hemagglutination, or hemadsorption (Example 1). Fecal samples are screened for evidence of parasites, such as toxoplasma.

After surgically harvesting the tissues of interest, a gross necropsy examination is performed on the donor pig by a certified pathologist. Selected tissue samples of respiratory, cardiovascular, hepatobiliary, blood, CNS tissue, gastrointestinal, urogenital, and hemolymphatic systems are preserved and archived for future reference.

Collection of Fetal Tissues

When fetal tissue is generated for transplantation, the list of pathogens tested is expanded to include pathogens capable of crossing the placental barrier and tissue-specific pathogens, e.g., neurotropic pathogens. Prior to artificial insemination, blood and fecal samples are collected from the gilt and tested for the presence of bacterial, fungal, or viral pathogens, as described above. A gilt essentially free of pathogens is artificially inseminated by methods known in the art and maintained under pathogen-free conditions as described above. At a designated time of gestation, a hysterectomy is performed under aseptic conditions. Fetuses are removed from their individual amniotic sacs and the desired fetal cells or tissues are thereafter excised and maintained under appropriate conditions to maintain viability and sterility.

Porcine Fetal Neuronal Cells for Transplantation into Parkinson's Disease Patients

Porcine fetal neuronal cells from pathogen-free fetal pigs were implanted into the putamen and caudate of patients with Parkinson's disease following the procedures described in Example 3. Subjects were selected for treatment from patients having advanced idiopathic Parkinson's disease of 7-20 years duration, and base-line clinical evaluations obtained. Fetal mesencephalic cells were implanted as described below, and patients evaluated post-operatively every three months over an initial three year period. Recipient patients are treated with immunosuppressive therapy (cyclosporin) or receive neuronal cells pretreated with F(ab′)₂ directed against MHC-Class I antigen. F(ab′)₂ immunomodulation treatment has been shown to induce graft acceptance (Pakzaban et al. (1995) Neuroscience 65:983-996). Other types of immunosuppressive or immunomodulation treatments may be used with the invention, for example cyclosporin, FK-506.

### EXAMPLE 1

### Testing Procedures

Virus Detection

Preparation and Maintenance of Cells Lines for Viral Co-Cultivations

The cell lines used to detect viruses in porcine cells include African Green Monkey kidney (VERO) (ATCC CC181), human embryonic lung fibroblasts (MRC-5) (ATCC CCL 171), porcine kidney (PK-15), (ATCC CRL 33), porcine fetal testis (PFT) (ATCC CRL 1746) and human glioblastoma cell line ATCC CRL 1690. These cell lines are passaged and maintained in cell cultures under standard cell culture conditions (Hsuing (1982) in Diagnostic Virology, Yale University Press, New Haven, Conn.; Daniel & Terr (1991) in Basic and Clinical Immunology, Appleton & Lange, East Norwalk, Conn.).

Inoculation of Cells Cultures with Samples and Controls

Negative controls consist of flasks containing cells in sterile cell culture medium. Positive controls include VERO and MRC-5 cells inoculated with poliovirus type 1 attenuated (ATCC VR-192) and measles virus, Edmonston strain (ATCC VR-24), swine influenza virus type A (ATCC VR-99), parainfluenza virus PI₃ (ATCC VR-281); PK-15 and ST cells inoculated with swine influenza type A (SIV) (ATCC VR-99), porcine parvovirus (ATCC VR-742), parainfluenza PI₃, and transmissible gastroenteritis of swine (ATCC VR-743); human glioblastoma cells are inoculated with pseudorabies (ATCC VR135).

Flasks of cultured cells are prepared and checked to ensure proper confluency (approximately 70-80%). Frozen samples to be tested are allowed to thaw. Positive control flasks are inoculated with approximately 100 TCID50 of each virus in a volume of 1 ml. Negative control flasks are prepared by removing media and adding 1.0 ml sterile media. For each cell line, test flasks are inoculated in triplicate with a minimum of 1.0 ml of sample material. Each inoculum is adsorbed for 1-2 h at 35-37° C. The blood or fetal cells are then removed, and the cell cultures refed with the fresh maintenance medium appropriate for that cell line.

Observations for Viral Cytopathic Effects (CPE)

All cultures are observed for viral CPE at least three times per week for a minimum of 21 days. Flasks are removed from the incubator and each cell culture is observed under an inverted microscope with 40× to 200× magnification. Any abnormalities in the cell monolayers are noted. If abnormalities are present, the supernatant is collected, and cells subcultured. After 7 and 14 days, two blind passages of each test sample are made by collecting supernatant and cells from all flasks inoculated with each sample.

After incubation for 21 days, the hemadsorption and hemagglutination tests described below are performed. Subcultures are also tested 21 days after subculturing. Viral isolates are identified based on the cell line in which growth occurred, characteristics of the viral CPE, and/or the hemadsorption (HA) and hemagglutination (HAd) reactions. Cultured cells are stained with fluorescent antibodies specific for rabies, pseudorabies, parvovirus, enterovirus, adenovirus, transmissible gastroenteritis virus, bovine viral diarrhea, encephalomyocarditis virus, porcine respiratory and reproductive syndrome, and vesicular stomatitis virus, as described below. Criteria for a valid test require no evidence of viral CPE, HA, HAd, or fluorescence in the negative control, and all positive control viruses must be detected in at least one cell line. A tested sample is considered positive for the presence of a viral agent if any of the cell lines used in the study show any sign of viral CPE, HA, HAd, or fluorescence in a valid assay.

Hemagglutination (HA) Test

Fresh suspensions of rhesus monkey, guinea pig, chicken and porcine erythrocytes are prepared in phosphate buffered saline (PBS) at a concentration of 0.5%. After 14 days incubation or at the end of the incubation period (21 d), 1 ml of culture fluid is collected and pooled from each flask exposed to the test material, and from the positive and negative control flasks. To each tube is added 0.1 ml of one of the erythrocyte suspensions. Each tube is capped and vortexed. One set of tubes is incubated at 2-8° C. until tight buttons of erythrocytes form in the negative control (approximately 30-60 minutes). A second set of test tubes is incubated at 35-37° C. until tight buttons form in the negative control (approximately 30-60 minutes). Results are indicated as follows: formation of a tight button of erythrocytes indicates a negative result, e.g., no detectable level of hemoagglutination viruses. A coating of the bottom of the tube with erythrocytes indicates a positive result.

Hemadsorption (HAd) Test

HAd is performed on one ore more monolayers of the different cell lines including positive and negative control cultures. Within 14 days of inoculation or at the end of the incubation period, the cell monolayers in the flasks are washed 1-2 times with 3-6 mls phosphate buffered saline (PBS). To each flask is added 1-2 ml of the appropriate erythrocyte suspension, and the erythrocytes allowed to remain in contact with the cell monolayers. The flasks are incubated at 2-8° C. for 15-20 min, and unabsorbed erythrocytes removed by shaking. Erythrocytes are observed by placing the flasks on the lowered stage of a microscope, and viewing them under low power magnification. A negative result is indicated by a lack of erythrocytes adhering to the monolayer. Adsorption of the erythrocytes onto the cell monolayer indicates the presence of a hemoadsorbing virus.

Direct Fluorescent Antibody Stain for Detection of Viruses

Fluorescent antibodies specific to the following viruses are commercially available: rabies, pseudorabies, parvovirus, enterovirus, adenovirus, transmissible gastroenteritis virus, bovine viral diarrhea, encephalomyocarditis virus, porcine respiratory and reproductive syndrome, and vesicular stomatitis virus.

Cultured cells (VERO, MRC-5, PK-15, PFT, and human glioblastoma cells) grown to a monolayer in a T25 flask are detached with 2.5 ml of a 0.05% trypsin solution. The cells are washed twice in 2.5 ml Hanks Balanced Saline Solution (HBSS) to neutralize trypsin activity, then diluted in 5 ml HBSS. The cells are counted in a hemocytometer. A ring slide is prepared for each fluorescent antibody, and a 50 μl aliquot of a 1×10⁴ cells/μl cell suspension placed in each ring of the microscope slide and allowed to air dry. The air dried slides are placed in a slide rack and immersed in acetone previously cooled for a minimum of 30 min in a −70° C. freezer. The slides are placed in the freezer for 30 min. Acetone is removed and cells are rehydrated with PBS. The cells are then covered with 50 μl of each type of fluorescent antibody (concentrations determined according to standard methods or per manufacturer's directions). The slides are then incubated in a humidifying chamber at 36° C. for 45 min. The slides are immersed in a container containing 500 ml HBSS, and the HBSS is stirred for 4-5 min. The wash is repeated in fresh HBSS. The slides are then immersed in water and the immersion repeated 5 times. The slides are then counterstained by immersion in Evan's Blue solution for 5 min at room temperature. The HBSS and water wash steps are repeated. The slides are allowed to air dry and inspected under a fluorescence microscope. Criteria for a valid test require that no evidence of inclusion bodies characteristic of viral infection should be observed in uninoculated negative control slides. Characteristic viral inclusions in the positive controls require that TGE, SIV, and parvovirus be detectable by fluorescent staining in at least one of the cell lines used.

Bacteria Detection in Blood Cultures. The following procedure describes a method for processing blood cultures in order to maximize detection of a bacterium contained therein (Manual of Clinical Microbiology 5th Ed. (1991) American Society for Microbiology, Ch. 3, pp. 5-107 and Ch. 20, pp. 1203-1225).

Blood specimens are collected into aerobic and anaerobic blood culture bottles. Under a laminar flow hood, the bottle tops are disinfected with 70% isopropyl alcohol followed by betadine. After drying, the tops are rewiped with 70% isopropyl alcohol. A negative control is prepared consisting of 5 ml of sterile saline in one aerobic bottle and 5 ml of sterile saline in one anaerobic bottle. Positive controls include an aerobic bottle for Bacillus subtilis and an anaerobic bottle for Bacteroides vulgaris. The rubber stopper and cap are removed from the aerobic bottle, and bottles are incubated in 5% CO₂ for 21 days at 35-37° C. Bottles are inspected daily for any signs of bacterial growth, e.g., gas bubbles, turbidity, discoloration or discrete lumps. If signs of bacterial growth are observed, a gram stain is prepared and viewed microscopically at 100× oil immersion for the presence of a bacteria or fungus. Yeast or fungi found with the gram stain are subcultured onto a Sabouraud dextrose/Emmons plate. Positive samples are subcultured onto both chocolate agar plates with Iso Vitlex and onto BMB plates. The chocolate plate is incubated at 35-37° C. in 5% CO₂ for 24 h; the BMB plate is incubated anaerobically at 35-37° C. for 48 h.

A Vitek Bacterial Identification System is used to identify bacteria and yeast. Fungi are identified via their macroscopic and microscopic characteristics.

If no signs of growth are observed at the end of 21 days, a gram stain is prepared and the sample observed microscopically for the presence of bacteria and fungi. Criteria for a valid test requires the absence of growth in the negative control bottles and the presence of growth in the positive control bottles. The absence of any signs of growth in both the aerobic and anaerobic blood culture bottles, as well as absence of organisms seen on gram stain indicates a negative blood culture.

Fecal Examination for Parasites

This procedure describes the processing of swine fecal samples and examination for presence of parasite eggs and protozoa.

Fecal samples are collected in a collection vial and processed within 1 h of collection. One part specimen is transferred to a vial with 3 parts fixative (polyvinyl alcohol)(PVA) and 10% buffered formalin. A formalin-ethyl acetate solution is prepared as follows: About 1.5 cm diameter of specimen is mixed in 10 ml 0.85% saline, and strained through a wire mesh strainer into a 15 ml conical centrifuge tube. The tube is centrifuged at 650×g for 2 min to sediment the remaining fecal material. The supernatant is carefully decanted, and 10% buffered formalin is added to the supernatant to a total volume of 9 ml. The solution is mixed, allowed to stand for 5 min, then 4 ml of ethyl acetate is added. The tube is capped and mixed vigorously in an inverted position for 30 . The cap is removed to vent the tube, then replaced. The tube is centrifuged at 500×g for 1 min. Four phases result (top to bottom): ethyl acetate, debris plug, formalin, and sediment. The debris plug is carefully rimmed using an applicator stick, and the top three layers discarded by pouring into a solvent container. The debris is removed using a cotton tipped applicator swab. The sediment is mixed with either a drop of formalin or the small amount of formalin which may remain in the tube after decanting.

Duplicate wet mounts are prepared by placing 2 drops of the sediment mixture on a clean 2″×3″ slide, adding a drop of 1% Lugol's iodine solution and a cover slip. The slide is examined microscopically under 10× objective, then under 40× objective. Any protozoan cysts of trophozoites and helminth eggs and larvae are identified. Protozoan cyst identification is confirmed by trichrome stain. The parasite screening procedures may also be conducted where appropriate through serological testing.

### EXAMPLE 2

Fetal Neuronal Tissue Isolation from Ventral Mesencephalon Animals: Qualification for Gilts

Gilts are screened for evidence of infection by serology, fecal parasitology, and a complete blood count with blood smear, as described above. Specific pathogens screened by serological testing are: toxoplasma, eperythrozoon suis, brucella, leptospirillum, mycoplasma hyopneumonia, porcine respiratory reproductive syndrome, pseudorabies, swine influenza type A, transmissible gastroenteritis, bovine viral diarrhea, encephalomyocarditis, vesicular stomatitis virus, and parvovirus. Further, gilts are tested for tuberculosis, vaccinated for erysipelas, parvovirus, and leptospira, and wormed with Ivermectin.

Qualification for Boar and Boar Semen

The boar and semen are qualified by serology screening, parasitology testing, CBC, TB screening, blood culture, and viral co-cultivation assays at six-month intervals. The list of viruses screened are the same as those described above for gilts. In addition, sperm is evaluated prior to each insemination for volume, total count, morphology, and motility.

Maintenance and Artificial Insemination

The following procedures describe the general maintenance and intermediate testing performed on the donor animals. After passing the serology screen, animals are maintained in a temperature and humidity controlled room on a 12 hours light/dark cycle under positive pressure with air purified through HEPA filters. Personnel entering this designated isolation area must be suited with coveralls, foot covers, gloves, and a mask. The estrous cycle of donor animals is manipulated by the administration of hormones and animals are artificially inseminated at the appropriate time. Approximately 32 days prior to a scheduled transplant, blood and fecal samples are collected from the gilt and screened for evidence of parasites. Viral pathogens which are zoonotic, capable of crossing the placental barrier, and affecting the tissue, cells or organ to be transplanted are screened at this time. Samples are co-cultivated on live indicator cells and assayed for hemagglutination, hemadsorption, cytopathic effect, and the presence of specific viral antigens by fluorescent antibody staining. All procedures are performed in a horizontal flow bench and Biological Safety Cabinet (BSC).

Uterine Harvest

After pregnancy is confirmed, the animal is sacrificed and surgical harvest of the uterus is performed in a sterile isolator. The surgically removed uterus is transferred to a BSC in a HEPA-filtered room, cut open and fetuses are dissected from the uterine horn, removed from their embryonic sacs, and placed in a dish of cold (4-10° C.) sterile Dulbecco's phosphate buffered saline (DPBS).

Fetal Dissection of Tissue For Treatment of Parkinson's Disease

The uterus and embryonic sacs are fixed in formalin and submitted to quality control (QC) for morphological examination by a certified pathologist. Fetuses are transferred to a fresh dish of cold sterile DPBS and the crown to rump length (CRL) is measured for each fetus. The acceptable size range of a fetus is 14-25 mm. Fetal heads are decapitated and two of the decapitated bodies and one intact body are submitted to QC for morphological examination. The fetal head is placed in a black saucer of cold sterile DPBS. Using forceps and microscissors, the ventral mesencephalon (VM) and dorsal mesencephalon (DM) are dissected from the fetal brain. Extraneous brain tissue is placed in a separate labelled 15 ml conical tube and submitted to QC for morphological examination.

VM fragments are transferred to a sterile 15 ml centrifuge tube. The fragments are allowed to settle and the HBSS is removed with a pipette. 1.5 ml of 0.05% trypsin-EDTA is added, and the tube incubated at 37° C. for 10±2 min. After removal of the trypsin-EDTA solution, the fragments are washed with 7 ml HBSS three times. One ml of HBSS containing 50 μg/ml DNase (Pulmozyme, Genentech) is added to the tube. The tissue is triturated with progressively smaller flame polished Pasteur pipets until a uniform milky suspension of single cells and small clumps of cells is obtained. A 5 μl aliquot is removed, stained with acridine-orange ethidium bromide (AO-EB), and cell number and viability count are performed. The cells are centrifuged at 500 rpm for 5 min. If cells are to be masked, the masking procedure is commenced at this point. If cells are not to be masked, the supernatant is removed and the cells are resuspended in a 0.9% saline-0.36% glucose solution at 50,000-100,000 cells/μl. Cell samples (12.3×10⁶ cells) are submitted to QC for testing. Cells are transferred to a capped tube and stored on ice until transplanted (10×10⁶ to 30×10⁶ cells for transplantation).

### EXAMPLE 3

Porcine Fetal Neuronal Cells for Transplantation into Parkinson's Disease Patients

Porcine fetal neural cells from pathogen-free fetal animals were implanted into the putamen and caudate of patients with Parkinson's disease using standard stereotaxic surgical techniques under local anesthesia, following the procedures described herein. Patients were selected for treatment as described below.

Patient Inclusion Criteria

Two patient groups were transplanted with fetal porcine neural cells. The first group was transplanted with cells in combination with standard immunosuppression (cyclosporin A). The second group was transplanted with fetal porcine neural cells pretreated with a F(ab)₂ fragment directed against MHC-Class I, which has been shown to induce graft acceptance.

Patients suitable for treatment have advanced Parkinson's disease of 7 to 20 years duration. In all patients, medical therapy has failed or begun to fail with signs of severe bradykinesia, dyskinesia, and marked on/off phenomena. Failed medical therapy is defined as patients who, despite the use of Sinemet and a dopamine agonist (bromocriptine or pergolide) or a combination of dopamine and selegiline, have approximately 25% of the time when they are “off” or have disabling dyskinesia approximately 25% of the time. Patients defined as in the Hoehn and Yahr stage 3 through stage 5 fit this definition (Hoehn & Yahr (1967) Neurology 17:427-442).

The Core Assessment Program for Intracerebral Transplantation (CAPIT) (Langston et al. (1992) Movement Disorders 7:2) is used to evaluate patients. Parkinsonism is idiopathic in nature and not due to tumors, infection, cerebrovascular disease, or trauma. Idiopathic Parkinsonism is determined by the presence of two of the following factors: 1) bradykinesia, 2) tremors, 3) rigidity, or 4) postural instability, when at least one of which is either tremor or bradykinesia. Patients are unequivocally responsive to L-dopa therapy by showing a 33% improvement in their Unified Parkinson's Disease Rating Scale (UPDRS) (Fahn et al., (1987) in Recent Developments in Parkinson's Disease, Vol. 2 (MacMillan Healthcare Information, Florham Park, N.J.); 153-163 and 293-304) score over that measured in their worst “off” as defined in CAPIT. Patients have intractable symptoms despite optimal therapy including frequent “off” episodes, disabling dyskinesias, or freezing while “on”. Patients are also Magnetic Resonance Imaging (MRI) negative, functionally defined as the absence of T2 hypointensity and lack of gross evidence of decreased width of the pars compacta or decrease in volume of subcortical nuclei.

Transplants are scheduled as frequently as once every 4 weeks, and the transplant patient evaluated by UPDRS within one week prior to the next scheduled transplantation.

Patient Exclusion Criteria

Patients are excluded from treatment on the basis of diagnosis of secondary Parkinson's as indicated by Parkinson “Plus” syndromes. Patients with mini-mental state score of 22 or less (maximum=30) are excluded to eliminate dementia, since dementia may indicate the presence of accompanying Alzheimer disease or diffuse Lewy body disease, which could interfere with tolerance to medication or the ability to adequately test the patient. Patients with a Hamilton Depression Scale of 20 or more points are excluded to eliminate patients with major depression, since depression could interfere with obtaining accurate UPDRS scores and global rating results. Patients are also excluded based on the presence of significant medical disease.

Evaluation of Patient Parkinsonism

Baseline—Core evaluation. Patients are evaluated by UPDRS as the primary clinical assessment scale. Ratings are also established according to Hoehn & Yahr Staging and Dyskinesia Rating Scale evaluation scale per CAPIT. Additionally, timed tests of motor function are performed, including pronation-supination test, hand/arm movement between two points, finger dexterity and stand-walk-sit test. Pharmacologic tests include single dose L-dopa test in defined “off” state as defined by CAPIT. Timed tests are videotaped to document disease state. MRI and Positron Emission Tomography (PET) scans are also conducted.

Because of the day-to-day variability in Parkinson's disease, patients are evaluated over a 1-3 month period to establish base-line clinical status. One to three separate evaluations are conducted, including all day observations. The actual number of evaluations is dependent upon whether or not the patient had previously been followed by the same neurological group for an extended period of time. Patient medication is kept constant during the observations. At each evaluation, blood samples are drawn for standard cellular and chemistry tests. In patients receiving cyclosporin, post-transplantation blood samples are monitored for cyclosporin levels.

Post-Operative Evaluations

Patients are followed for three years post-operatively, with Core Evaluations conducted every three months. Follow-up MRI evaluations are performed 1 week and 6 months post-operatively. Follow-up PET scans are conducted at 6, 12, 18, and 24 months postoperatively. After the initial three year follow-up period, patients are evaluated on an annual basis.

Type, Number, and Concentration of Cells Implanted

Fetal mesencephalic cell suspensions are prepared from dissection of the rostral half of the ventral mesencephalon of porcine embryonic tissue from E23-28 (days since conception/fertilization of oocytes) aged fetuses. Time-mated, ultrasound confirmed pregnant Yorkshire pigs are euthanized according to the standard veterinary procedures of Tufts University School of Veterinary Medicine. The ventral mesencephalon from the fetuses are carefully dissected under microscopic guidance, then pooled, incubated and trypsinized to prepare a cell suspension for transplantation. Cells are prepared at a concentration of 50,000-100,000 cells/μl, and are assessed for viability as follows. Cells are mixed (1 part cells:4 parts dye, usually 5 μl cells+20 μl dye) with acridine orange/ethidium bromide dye (0.01% each dye in PBS) then a hemacytometer is loaded with 20 μl of the cell/dye suspension. The same is then viewed in a fluorescence microscope with fluorescein filters, under which live cells appear green and dead ones orange.

40 μl volumes of cell suspensions are implanted at each of six to eight stereotaxic targets in the putamen and caudate on one side. Thus, a total cell suspension volume of between 240 μl-480 μl may be injected.

Prior experiments show that about 10% of cells survive implantation. Therefore, 10×10⁶ to 30×10⁶ injected cells are expected to yield approximately 10×10⁵ to 30×10⁵ surviving cells, of which about 10% are mesencephalic dopaminergic cells. Consequently, 10×10⁴ to 30×10⁴ dopaminergic cells are replaced by transplantation. In advanced Parkinson's disease, patients have lost at least 80-90% of 25×10⁴ dopaminergic cells normally providing dopamine to the putamen, and 60-70% of 25×10⁴ cells providing dopamine to the caudate.

Implantation Sites and Procedure

Based on the specific neuroanatomical architecture of an individual patient, a minimum of two and a maximum of four sites in the caudate, and a minimum of four and a maximum of eight sites in the posterior putamen are targeted for transplantation. All patients undergo unilateral stereotaxic implantation of cells using MRI guided technique. The CRW stereotaxic frame utilized for this procedure is routinely used for precise targeting of structures in the brain for biopsies or functional ablation therapy. The procedure uses a cannula (Radionics Co., Burlington, Mass.) with an outer diameter of 1.0 mm, and inner diameter of 0.5 mm. A micromanipulator apparatus is used in conjunction with the stereotaxic frame localizer. Specific locations in the putamen and caudate are determined in each patient by MRI. Under sterile conditions, a 3 cm incision is made in the right front scalp. A burr hole is made in the calvarium, 2 cm lateral to the midline and 1 cm anterior to the coronal suture. The meninges are opened with a coagulator to allow safe and smooth penetration of the catheter. Between three and six needle entries are made into the brain of each patient: one or two in the caudate head and two to four in the posterior putamen. The needle passes into the putamen are spaced to evenly distribute cells over the region. Up to 40 μl of cells are injected first at the deepest target site approximately 5 mm from the ventral limits of the putamen and caudate head. After injection of the cells, the needle is withdrawn approximately 5 mm to a more superficial location where the second up to 40 μl of cells are injected. The results is a column of cells extending from the deepest point, back along the needle tract to the dorsal edge of the putamen and caudate head. After the final injection of cells, the injected cell suspension is allowed to equilibrate over an eight minute period so that there is no movement of cells along the needle tract as the needle is removed. Along the anterior - posterior axis the putamen needle tracts are space 4 mm from each other with the most posterior tract being 4 mm from the posterior limit of the putamen. If two tracts are made in the caudate, the tracts are spaced equidistant from each other.

During the entire procedure, the patient is awake and receives only subcutaneous lidocaine in the scalp and mild intravenous sedation. Since the brain lacks pain receptors, the passage of the catheter and the transplantation procedure are painless. At the end of the transplantation procedure, the scalp incision is closed with nylon sutures, a sterile dressing applied and the stereotaxic frame removed. The patient is kept under observation for 24 hr; normal activities and eating are resumed on the first post-operative day, and patients in good condition are discharged after 2 days.

Immunosuppression

In patients receiving immunosuppressive therapy, the first dose of cyclosporin is given 12 hr prior to transplantation as a single 15 mg/kg dose, and daily doses are given in the range of 14-18 mg/kg, based on dosages routinely used in major organ transplants. The dose of cyclosporin is reduced to a maintenance level of 5-10 mg/kg/day 3-6 months post-transplantation when the blood-brain barrier is reclosed, based on evidence that the brain provides a relatively immunopriviledged site. Serum levels of cyclosporin are monitored on a regular basis during patient follow up visits, as described above. The desired serum level for cyclosporin is 100-150 ng/ml.

F(ab′)₂ Immunomodulation

A group of patients receive fetal porcine neural cells pretreated with an F(ab′)₂ directed against MHC-Class I. The F(ab′)₂ fragment is manufactured from a mouse monoclonal antibody. This immunomodulation treatment has been shown to induce graft acceptance in animal models (Pakzaban et al. (1995) supra). No adverse effects have been seen with the F(ab′)₂ immunomodulation treatment in animal systems.

Results

Clinical studies of fetal porcine neuronal cell transplantation into patients suffering severe Parkinson's disease were conducted as described above. To date, four patients have been transplanted of the 12-patient study. The first three transplant recipients received cyclosporin, while the fourth recipient received transplanted neuronal cells treated to prevent rejection. All four recipients have exhibited positive results, and the first patient's seven month evaluation exhibited significant improvement in PET scan results.

What is claimed is:

1. An enclosure containing two or more pigs, wherein each pig is essentially free of pathogens.

2. The enclosure of claim 1, wherein said pig is free of zoonotic pathogens.

3. The enclosure of claim 2, wherein said zoonotic pathogens are brucella, encephalomyocarditis virus, leptospirillum, listeria, mycobacterium TB, pseudorabies virus, rabies virus, swine influenza type A virus, toxoplasma, transmissible gastroenteritis virus, and vesicular stomatitis virus.

4. A method for producing and maintaining a pig as a source of tissues, cells or organs for transplantation into a human recipient, wherein said pig is free of pathogens, said method comprising:
selecting a pig free of pathogens; and
maintaining said pig in an enclosure under conditions whereby said pig remains free of pathogens.

5. The method of claim 4, wherein said pig is free of zoonotic pathogens.

6. The method of claim 5, wherein said zoonotic pathogens are brucella, encephalomyocarditis virus, leptospirillum, listeria, mycobacterium TB, pseudorabies virus, rabies virus, swine influenza type A virus, toxoplasma, transmissible gastroenteritis virus, and vesicular stomatitis virus.

7. A method of providing tissues, cells, or organs for transplantation into a human recipient in need thereof, said method comprising:
a) providing a pig free of zoonotic pathogens and pathogens affecting said tissues, cells, or organs;
b) harvesting said tissues, cells, or organs from said pig; and
c) transplanting said tissues, cells, or organs into said recipient.

8. The method of claim 7, wherein pancreatic islet cells are provided for transplantation into a human recipient with islet insufficiency, wherein said pig is free of zoonotic pathogens and pathogens affecting said pancreatic islet cells, said islet cells are harvested from said pig and transplanted into said recipient.

9. The method of claim 8, wherein said zoonotic pathogens and pathogens affecting said pancreatic islet cells include adenovirus, ascarids, bovine viral diarrhea, brucella, clostridium, echinococcus, encephalomyocarditis virus, eperythrozoon parvum, eperythrozoon suis, hemagglutinating encephalomyocarditis, haemophilus suis, leptospirillum, listeria, mycobacterium TB, mycoplasma hyopneumonia, parvovirus, porcine respiratory reproductive syndrome virus, pseudorabies virus, rabies virus, salmonella, suipoxvirus, swine influenza type A virus, teschen, toxoplasma, transmissible gastroenteritis virus, and vesicular stomatitis virus.

10. The method of claim 7, wherein hepatocytes for are provided transplantation into a human recipient, wherein said pig is free of zoonotic pathogens and pathogens affecting hepatocytes, said hepatocytes are harvested from said pig and transplanted into said recipient.

11. The method of claim 10, wherein said recipient suffers from liver failure or insufficient liver function.

12. The method of claim 7, wherein keratinocytes are provided for transplantation into a human recipient in need thereof, wherein said pig is free of zoonotic pathogens and pathogens affecting keratinocytes, said keratinocytes are harvested from said pig and transplanted into said recipient.

13. A method for producing fetal porcine tissues, cells, or organs for transplantation into a human recipient, said method comprising:
selecting a female pig which is free of zoonotic pathogens, pathogens capable of crossing the placental barrier, and pathogens affecting the tissues, cells, or organs to be transplanted;
maintaining said pig in an enclosure under conditions whereby said pig remains free of said pathogens;
artificially inseminating said pig; and
harvesting said tissues, cells, or organ from a fetus of said pig wherein said tissues, cells, or organ are free of said pathogens.

14. The method of claim 13, wherein said zoonotic pathogens and pathogens capable of crossing the placental barrier include brucella, eperythrozoon suis, encephalomyocarditis virus, hemagglutinating encephalomyocarditis virus, leptospirillum, listeria, mycobacterium TB, mycoplasma hyponeumonia, parvovirus, pseudorabies virus, porcine respiratory reproductive syndrome virus, rabies virus, suipoxvirus, swine influenza type A virus, swine vesicular disease virus, teschen, toxoplasma, transmissible gastroenteritis virus, and vesicular stomatitis virus.

15. The method of claim 13, wherein fetal porcine neuronal cells are produced for transplantation into a human recipient, wherein said female pig is free of zoonotic pathogens, pathogens capable of crossing the placental barrier, and neurotropic pathogens.

16. The method of claim 15, wherein said neurotrophic pathogens include adenovirus, encephalomyocarditis virus hemagglutinating encephalomyocarditis, listeria, parvovirus, pseudorabies, rabies, swine vesicular virus, teschen, and toxoplasma.

17. The method of claim 15, wherein said patient suffers from one of the following conditions or diseases: Parkinson's disease, Huntington's disease, stroke, focal epilepsy, Alzheimer's disease, amyotrophic lateral sclerosis (ALS), spinal cord injury, and cognitive disorders.

18. The method of claim 13, wherein fetal porcine cardiac myocytes are produced for transplantation into a human host, wherein said female pig is free of zoonotic pathogens, pathogens capable of crossing the placental barrier, and pathogens affecting cardiac myocytes.

19. The method of claim 13, wherein fetal porcine pancreatic islets are produced for transplantation into a human host, wherein said female pig is free of zoonotic pathogens, pathogens capable of crossing the placental barrier, and pathogens affecting pancreatic islets.

20. The method of claim 13, wherein fetal porcine hepatocytes are produced for transplantation into a human host, wherein said female pig is free of zoonotic pathogens, pathogens capable of crossing the placental barrier, and pathogens affecting hepatocytes.
